(19) United States
(12) Patent Application Publication
(10) Pub. No.: US 2016/0000564 A1
(43) Pub. Date: Jan. 7, 2016

(54) DELIVERY SYSTEMS FOR CARDIAC VALVE SUPPORT DEVICES

(71) Applicant: MVALVE TECHNOLOGIES LTD., Herzliya (IL)

(72) Inventors: Maurice BUCHBINDER, La Jolla, CA (US); Amit TUBISHEVITZ, Tel Aviv (IL); Shay DUBI, Tel Aviv (IL); Remo ALMOG, Tel Aviv (IL)

(21) Appl. No.: 14/768,588

(22) PCT Filed: Feb. 19, 2014
(86) PCT No.: PCT/IL2014/050183

§ 371 Date: Aug. 18, 2015

Related U.S. Application Data
(60) Provisional application No. 61/766,768, filed on Feb. 20, 2013.

(51) Int. Cl. A61F 2/24

(52) U.S. Cl. CPC A61F 2/2436

(57) ABSTRACT

The present device provides a delivery device (10) suitable for delivering a cardiac valve support device (11) that comprises at least one support element (14, 19) and two or more stabilizing elements (16), wherein said delivery device comprises a proximal handle and an outer conduit (12) that is continuous therewith, and wherein said delivery device further comprises various means for controlling the release of said support element from the open distal end of said outer conduit and means for retaining the support device stabilizing elements in a closed conformation, and independently controlling their release.

### FIELD OF THE INVENTION

The present invention is directed to devices for use in the minimally invasive delivery of cardiac valve support devices. More specifically, the present invention provides delivery devices that enable the delivery of cardiac valve support devices by the transapical and transseptal routes.

### BACKGROUND OF THE INVENTION

The present inventors have previously described a two-step method for replacing cardiac valves, in which the first step involves the delivery of a support device having at least one, annular-shaped support element, the outer rim of which becomes pressed against the cardiac tissue in the region of the valve annulus. In the second step, a cardiac valve prosthesis is delivered into the internal space bounded by the inner diameter of the support element and allowed to expand such that in its expanded conformation, said prosthesis becomes supported by said support device. A support device having two annular support elements connected by bridging elements is disclosed in co-owned, co-pending U.S. application Ser. No. 13/224,124, which published as US 2012/0059458. Another type of cardiac valve support device comprising a single annular support element is disclosed in co-owned PCT application no. PCT/1L2013/000025, which published as WO 2013/128436.

Various different approaches may be used to deliver a prosthetic cardiac valve (and any associated support elements) as part of a valve replacement procedure. It is to be noted that in most cases, prior art valve replacement procedures have necessitated the use of open-heart surgery, in which it is necessary to place the patient on cardiopulmonary bypass. One of the key advantages of both the support devices and of the valve-replacement methods disclosed in the aforementioned publications is that both said support device and the prosthetic valve that is supported thereby may be delivered percutaneously (for example, by the transapical route or the transseptal route) by means of crimping said devices such that they may be loaded into a catheter or other small-diameter delivery conduit, thereby obviating the need for more invasive open-heart surgery. Additionally, it should be noted that while prior art delivery systems were developed for the purpose of delivering a stent-like structure (generally defined as a tubular metallic mesh structure, which is crimped in a symmetrical radial position, and has a very small surface area in the radial plane—determined by the thickness of the material, but a significant longitudinal length, determined by the design), the support device mentioned herein is essentially an annular shaped ring; in sharp contrast with stents the support device has a very small longitudinal length—determined by the thickness of the material, and a significant surface area in the radial plane, determined by the design. Hence the annular support device cannot crimp in a symmetrical radial manner, and delivery systems known in the literature cannot, therefore, be used to deliver this system in a controllable and precise manner. In the case of transseptal delivery, the crimped valve support device is transported through the peripheral circulation (e.g. via the femoral or subclavian veins), by means of making small incisions in the skin and blood vessel wall. In the transapical approach, however, the crimped device is loaded into a rigid or semi-rigid small-diameter delivery conduit and passed via a small skin incision through an intercostal space such that it may be advanced through a puncture made in the heart muscle in the vicinity of the cardiac apex, into a ventricular cavity.

In the present case, in order to be able to bring both the support device and the replacement valve into their correct working locations, it is necessary to provide suitable small-profile delivery devices which are able to securely transport both of those elements in a collapsed or crimped state, and which are further capable of controllably releasing said elements, such that they are able to expand into their working conformation at the desired location.

In many embodiments of the valve support device developed by the present inventors, said device is fitted with a plurality of laterally-disposed arms, wings or other stabilizing elements, the purpose of which is to enable said device to become firmly anchored at its working site in the region of the anatomical valve annulus, and to resist displacement by the forces exerted by the beating heart. Examples of such stabilizing elements may be found in co-owned, co-pending international patent application no. PCT/IL2012/000093, filed on Feb. 28, 2012, and co-owned, co-pending U.S. patent application No. 61/752,994, filed on Jan. 16, 2013.

It may be appreciated that if said stabilizing elements were to be allowed to expand in a passive, uncontrolled manner at the same time as the aforementioned annular support elements and/or bridging elements adopt their expanded conformation (i.e. upon their release from the delivery device), it may not be possible to correctly orientate and anchor the support device. Rather, it is important that the operator is able to selectively delay the deployment (lateral expansion) of the stabilizing elements until after he or she has maneuvered the support device into its correct working position.

A need thus exists for a new delivery device that allows the operator to precisely control both the expansion and deployment of the crimped valve support structures, and, independently, the lateral expansion of the valve support stabilizing elements. The presently-disclosed delivery device fulfills this need.

### SUMMARY OF THE INVENTION

The present invention is, therefore, primarily directed to a delivery device suitable for delivering a cardiac valve support device fitted with at least one support element and two or more stabilizing elements, wherein said delivery device comprises, at its proximal end, a handle which is intended to remain outside of the patient's body, said handle being continuous at its distal end with an outer conduit having an internal diameter suitable for containing a cardiac valve support device in a first, collapsed conformation, and wherein said delivery device further comprises:
a) means for controlling the release of said support element(s) from the open distal end of said outer conduit;
b) means for retaining said support device stabilizing elements in a closed conformation, even after said support element has been released from the outer conduit; and
c) means for independently controlling the release of said stabilizing element retaining means, thereby permitting the lateral expansion of said stabilizing elements.

The term “independently controlling the release of said retaining means” is to be taken to refer to the fact that said release of the stabilizing element retaining means may be caused independently of the release of the support elements.

In certain embodiments of the presently-disclosed delivery vice, the means for retaining the stabilizing elements and the means for controlling the release of said stabilizing elements are provided by separate elements or structures. In other embodiments, the same element, structure or mechanism may be used to both retain the stabilizing elements and to control their release.

It is to be noted that the above-defined delivery device is suitable for use with any cardiac valve support device that comprises at least one support element (such as a ring-like structure) and two or more stabilizing elements. The latter term refers to any structure that arises from the support element(s) or is attached thereto, which may be used to stabilize or anchor the valve support device within the cardiac tissue. In many cases, said stabilizing elements are constructed in the form of “wings” or “arms” that curve away from their origin on the support element, ending in a free extremity that is used to make contact with the cardiac tissue. Many different types of such stabilizing element may be used to construct valve support devices that are suitable for delivery by means of the presently-disclosed device, and non-limiting examples may be found in the following co-owned patent documents, the contents of which are incorporated in their entirety into the present invention. U.S. patent application Ser. No. 13/790,174, published as US2014/0005778, international patent application number PCT/IL2013/000025, published as WO 2013/128436 and international patent application number PCT/IL2013/000036, published as WO2013/150512.

In the description that follows the term “stabilizing element” is used interchangeably with terms such as “wing”, “stabilizing arm”, and so on.

It should also be noted that some embodiments of the present invention will be described, and illustrated in the accompanying drawings, with regard to their use in the delivery of two-ring valve support devices, while other embodiments are shown in relation to the delivery of one-ring support devices. It is important, however, to appreciate that all of the embodiments of the delivery device described herein may be used to delivery either two-ring or single ring devices.

The terms “distal” and “proximal” as used herein in relation to the delivery device refer, respectively, to directions away from the operator and towards the operator.

As explained hereinabove, a key technical problem that is solved by the present invention relates to the need to selectively, and separately control the release of (i) the ring-like support element(s); and (ii) the generally elongate stabilizing wings or arms. Both of these elements are folded or “crimped” into the confines of the delivery device of the present invention, and it is essential that the operator be able to accurately control the deployment (unfolding) of said elements, in order to ensure correct implantation of the valve support device at its intended working position within the heart.

In one preferred embodiment of the device the means for controlling the release of the support element(s) from the outer conduit comprise an inner tube or rod located within the lumen of said conduit, and a mechanism for moving said inner tube or rod in a distal or proximal direction, said mechanism being operated by a rotatable handle or other suitable control element fitted on to the proximal handle of the delivery device. In another preferred embodiment, said means comprise a mechanism for moving the outer conduit distally or proximally (i.e. in relation to the inner tube or rod).

In another preferred embodiment of the device, he means for controlling the release of the support element(s) from the outer conduit comprise two or more pivotable jaws attached to the distal end of the inner tube or rod.

In another preferred embodiment, the means for controlling the release of the support element(s) from the outer conduit comprise a wire or thread.

In one preferred embodiment of the invention, the means for retaining the support device stabilizing elements in a closed conformation comprise a wire or thread. In one preferred implementation of this embodiment, said wire or thread has a first end held within the proximal handle or beyond the proximal end thereof, and said wire or thread passes distally from said first end and through the support device and then passes proximally to a second end held within the proximal handle or beyond the proximal end thereof, such that said stabilizing elements are retained in a closed, collapsed conformation by said wire or thread.

In one preferred embodiment, the means for controlling the release of said wire or thread retaining means comprises a pin or screw retaining one or both ends of said wire or thread within said proximal handle, wherein said pin or screw is capable of being manipulated such that it releases one or both ends of said wire or thread therefrom, thereby permitting one or both ends of said wire or thread to be withdrawn proximally, such that the stabilizing elements are allowed to adopt their laterally-expanded conformation.

In another preferred embodiment, the means for retaining the support device stabilizing elements in a closed conformation comprise two or more pivotable jaws attached to the distal end of the inner tube or rod. In this embodiment, the means for controlling the opening or closure of said jaws comprise a control element (such as a rotatable sleeve) situated in the proximal handle and a coupling element connecting said control element with said jaws, wherein said coupling element may be selected from the group consisting of one or more wires, one or more pusher rods and one or more rotatable threaded rods.

In one preferred embodiment of the invention, both the support element(s) and the stabilizing elements of the valve support device are retained by means of wires or threads, and selectively released therefrom by means of control elements in the proximal handle that permit said wires or threads to be either locked or released.

In one preferred embodiment of the invention, the inner tube is a multi-lumen tube.

In one preferred embodiment of the invention, the means for retaining the stabilizing elements in their closed conformation and also for controlling the opening and/or release comprise a laterally-expandable mechanism operated by a pusher tube, wherein said pusher tube is disposed co-axially with respect to the outer conduit.

In one particularly preferred embodiment of this aspect, the laterally-expandable mechanism is a hinged four-sided mechanism comprising:
a) two proximal movable arms and two distal movable arms joined together by means of pivotable junctions between two adjacent arms, such that said movable arms are capable of defining a quadrilateral outline shape;
b) two or more stabilizing element attachment arms pivotably attached at one of their ends to at least two of said pivotable junctions, wherein each of said short arms is adapted for attachment of a valve support device stabilizing element to its free end;
wherein the pusher tube is connected at its proximal end to a control mechanism within the proximal handle that may be used to move said tube proximally and distally;
and wherein said pusher tube is connected at its distal end to the pivotable junction between the two proximal movable arms.

In one preferred embodiment of the device, the outer conduit is rigid or semi-rigid, and wherein said device is suitable for use in a transapical procedure.

In another preferred embodiment of the device, the outer conduit is flexible, and wherein said device is suitable for use in a transseptal procedure.

In a particularly preferred embodiment of the present invention, the delivery device is suitable for the delivery of a mitral valve support device to the anatomical mitral annulus. However, in other preferred embodiments, the device may be used to deliver a support device to other locations in the heart, such as the aortic valve.

### BRIEF DESCRIPTION OF THE DRAWINGS

FIG. 1 schematically depicts the key features of the delivery device of the present invention.

FIG. 2 illustrates the external features of an exemplary transapical delivery device of the present invention.

FIG. 3 presents a longitudinal section view of the embodiment of the delivery device shown in FIG. 2.

FIG. 4 depicts an alternative embodiment of the transapical delivery device of the present invention, featuring a distally-located rotatable control sleeve and control wire release button.

FIG. 5 provides a longitudinal section view of the embodiment of the delivery device shown in FIG. 4.

FIG. 6 illustrates a modification of the embodiment shown in FIG. 5, in which the release button is located at the proximal extremity of the delivery device handle.

FIG. 7 depicts a further embodiment of the transapical delivery device of the present invention. A crimped valve support device is seen within the lumen of the outer conduit of the device.

FIG. 8 illustrates another embodiment of the transapical delivery device of the present invention, in which the proximal handle incorporates a marker pin located within a slot.

FIG. 9 shows an embodiment similar to that presented in FIG. 8, but further comprising a distal tip.

FIG. 10 illustrates a further embodiment of the device, featuring a jaw mechanism for retaining the support ring of a valve support device.

FIG. 11 depicts a transapical delivery device of the present invention with a valve support device held at the distal end of said delivery device by means of pivotable arms which grasp the stabilizing elements of said support device.

FIG. 12 provides an enlarged view of the distal end of the embodiment of the delivery device shown in FIG. 11, featuring a pair of pivotable jaws with curved free ends.

FIG. 13 schematically depicts the key features of the transseptal delivery device of the present invention.

FIG. 14 presents an external view of an exemplary transseptal delivery device of the present invention.

FIG. 15 presents an enlarged view of a transseptal delivery device following partial deployment of a two-ring cardiac valve support device.

FIG. 16 shows the embodiment of FIG. 15 at a later stage of the delivery procedure, following release of the entire support device from the catheter body.

FIG. 17 presents an alternative embodiment of the transseptal delivery device of the present invention, in which said device is fitted with a hollow proximal extension of the distal tip.

FIG. 18 shows a transapical delivery device in its pre-deployed configuration, wherein said device comprises a central multi-lumen tube having six peripheral lumens fir the control wires, and a central lumen for a guidewire.

FIG. 19 depicts the embodiment shown in FIG. 18 in its post-deployment configuration.

FIG. 20 is a photographic representation of the embodiment depicted in FIGS. 18 and 19, in which a single-ring valve support device is mounted on the distal end thereof.

FIG. 21 depicts a single-ring valve support device following its release from the delivery device shown in FIGS. 18 to 20, in which the stabilizing elements are in their fully-deployed configuration.

FIG. 22 shows the proximal end of one embodiment of the delivery device of the present invention, in which said device is fitted with a control wire tensioning mechanism.

FIG. 23 provides an external view of the same embodiment of the delivery device as shown in FIGS. 18 to 22, demonstrating the positions of the various control elements.

FIG. 24 presents an enlarged view of a mechanical stabilizing element deployment mechanism, said mechanism comprising four interconnected movable arms, the distal-proximal position of which is controlled by means of a pusher tube.

FIGS. 25-28 present the stabilizing element deployment mechanism depicted in FIG. 24, at various stages of the deployment, with FIG. 25 showing the mechanism in its initial position, and FIG. 28 illustrating the final, fully-deployed configuration.

FIG. 29 provides a diagrammatic representation of the angle between the short arm and the connected movable arm (of the mechanism shown in FIG. 24), and of the corresponding position of the stabilizing element that is attached to said short arm.

FIG. 30 provides a similar diagrammatic representation to that seen in FIG. 29. In this figure, however, the short arm, movable arm stabilizing element are all shown in their fully-deployed positions.

### DETAILED DESCRIPTION OF REFERRED EMBODIMENTS

### General Features of the Delivery Device of the Present Invention:

The features and advantages of the present invention will now be discussed with regard to the various embodiments shown in the appended drawings.

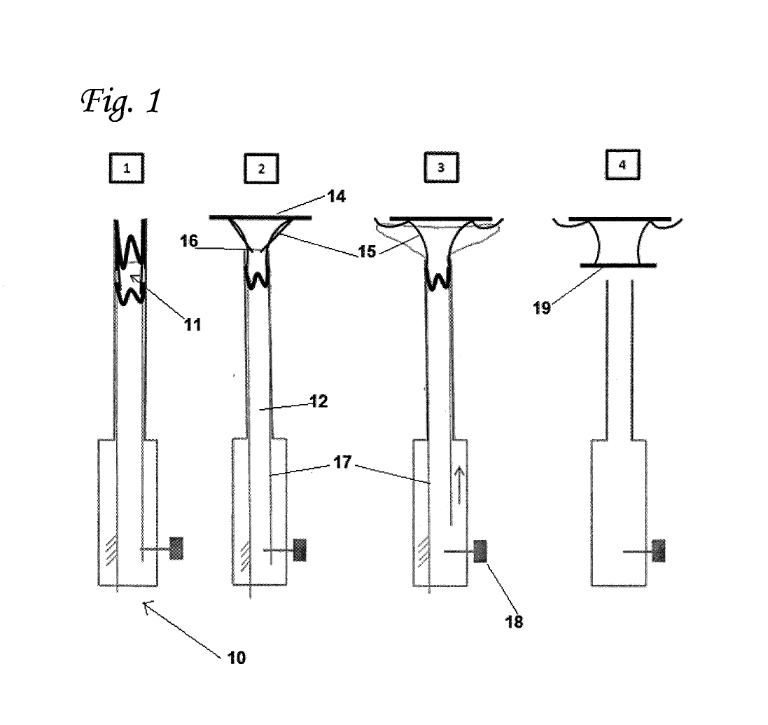

FIG. 1 schematically depicts, in a generalized manner, the key features of the presently-disclosed delivery device 10, and its use in the delivery of a two-ring valve support device 11. Four separate representations of the device are shown, each of which depicts a different stage in the deployment of the valve support device. Thus, in stage 1 (at the extreme left of the figure), the valve support device 11 is entirely contained within the outer conduit 12 of the delivery device. In stage 2, the upper support element 14 has been released from the confines of the outer conduit 12 and has adopted its expanded configuration. Similarly, bridging elements 15 have also been partially released. At this stage, stabilizing elements 16 are still retained in their collapsed configuration by means of stabilizing element control wire 17. Next, in stage 3, the control wire release screw 18 is manipulated in order to release the end of the control wire that is attached thereto, thereby causing said wire to lose its tautness and permitting stabilizing elements 16 to expand laterally into their working configuration. Finally, in stage 4, stabilizing element control wire 17 is removed from the device, and the lower support element 19 and the remaining portion of the bridging elements 15 are released from the outer conduit and allowed to expand into their final working configuration.

### Delivery Device for Transapical Use:

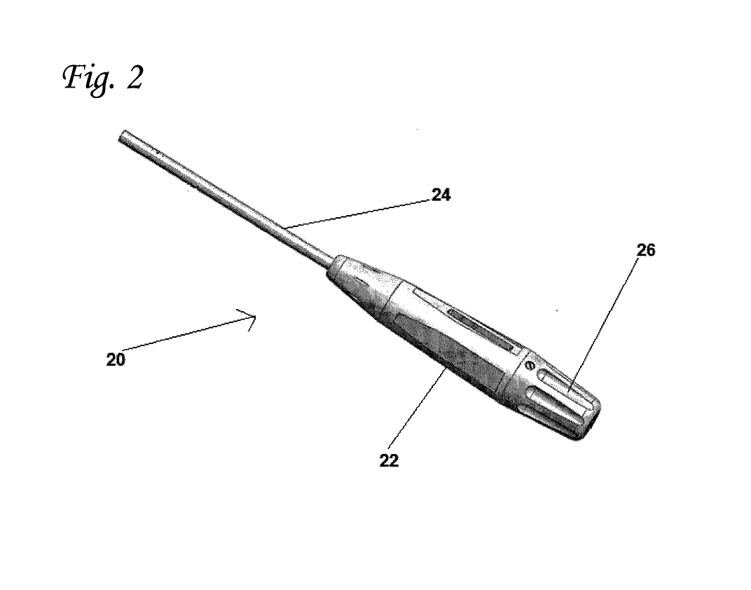

FIGS. 2 to 12 illustrate various preferred features of embodiments of the present device that are intended for use in the transapical delivery of a cardiac valve support device.

The external features of an exemplary transapical device 20 of the present invention are shown in FIG. 2. Thus, proximal handle 22 is connected to, and continuous with, the distally-placed outer conduit 24. The proximal extremity of handle 22 is formed as a rotatable control section 26 that is connected via an internal mechanism to either outer conduit 24 or to an inner tube or rod (not seen in this figure). In some preferred embodiments, rotation of this proximal portion of the handle causes movement of outer conduit 24 in relation to the inner tube or rod. This embodiment is particularly advantageous for the reason that retraction of the outer conduit in relation to the inner tube or rod does not alter the position of the valve support device in relation to the target deployment site (i.e. the anatomical valve annulus) and thus uniquely enables accurate deployment of the valve support device at an exact location. In other embodiments, however, a reverse approach is used, that is, rotation of this portion leads to movement of the inner tube or rod in relation to outer conduit 24. In either case, rotation of proximal control portion 26 will lead to a change in the distance between the distal tip of outer conduit 24 and the distal tip of the inner tube or rod. Consequently, when this distance is reduced, a crimped valve support device contained within the distal portion of the lumen of the outer conduit will be progressively exposed beyond said distal tip, and thereby allowed to expand.

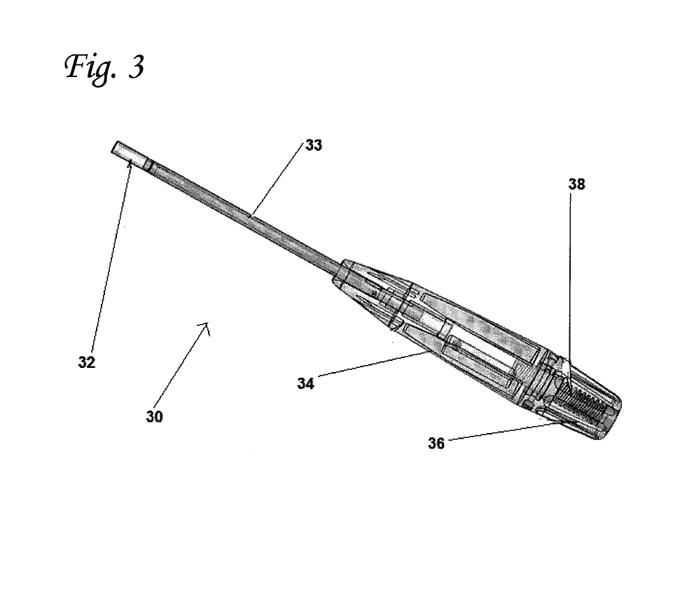

FIG. 3 is a longitudinal section view of the same embodiment shown in FIG. 2. It may be seen from this figure that the outer conduit 32 of delivery device 30 contains within its lumen an inner tube 33. Said outer conduit and inner tube each has a free distal end and a proximal end contained within proximal handle 34. The rotatable control portion 36, located at the proximal end of handle 34 contains a screw mechanism 38 which is connected to outer conduit 32. The valve support device to be delivered is crimped and then inserted into the lumen of outer conduit 32, distal to the free distal end of inner tube 33. After the distal tip of the device has been advanced to the desired target at the valve annulus, the rotatable control portion 36 is rotated in a direction that will cause outer conduit 32 to move proximally in relation to inner tube 33, thereby causing the support device to become progressively released from the confines of said outer conduit.

It is to be noted that for the sake of clarity, and in order to demonstrate some of the other key features of the device, neither FIG. 2 nor FIG. 3 show any of the elements involved in the retention of the stabilizing element in its closed conformation, or in the controlled expansion of said element.

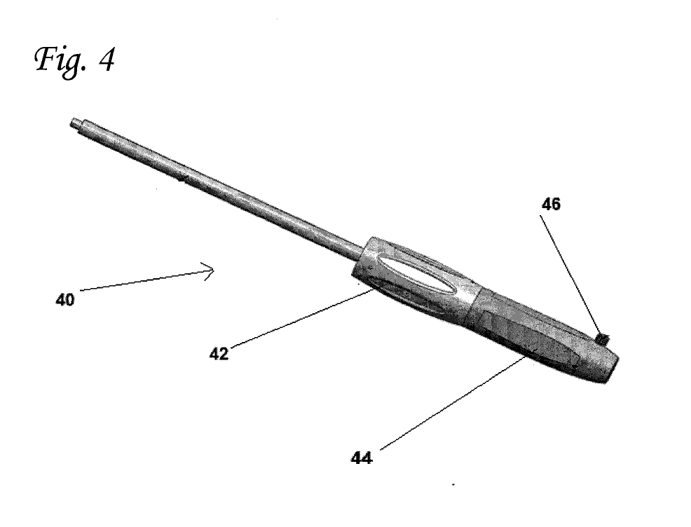

FIG. 4 depicts an alternative embodiment of the device of the invention 40 in which the rotatable control portion 42 of the handle is located distally to the fixed portion of the control handle 44. Also shown in this figure is a release button 46, for releasing and/or locking the control wire that retains the stabilizing elements of the valve support device in their closed (collapsed) configuration.

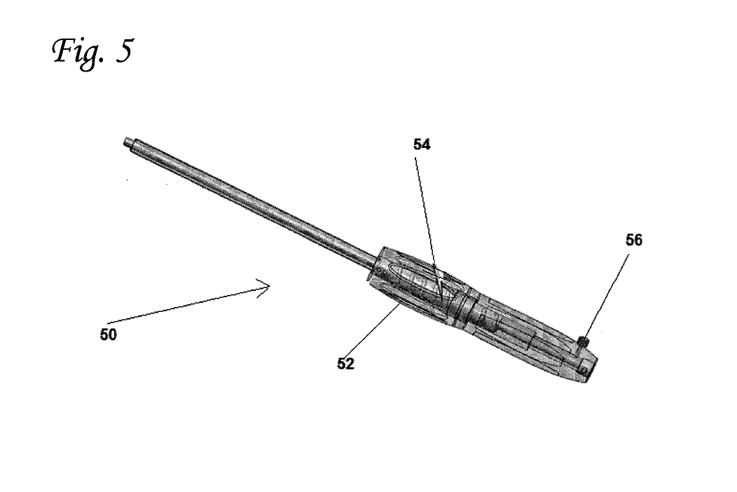

FIG. 5 presents a longitudinal section view of the same embodiment shown in FIG. 4. Thus, it may be seen from this internal view of delivery device 50 that the rotatable control portion 52 of the proximal handle overlays, and is connected to, an internal screw mechanism that when operated (by means of rotating control portion 52) causes the outer conduit to move either distally or proximally in relation to the inner tube. Control wire release button 56 is connected to a pin around which one end of the stabilizing element control wire (not shown) is wound. Manipulation of this button causes either release of the wire from said pin, or locking of the wire thereon.

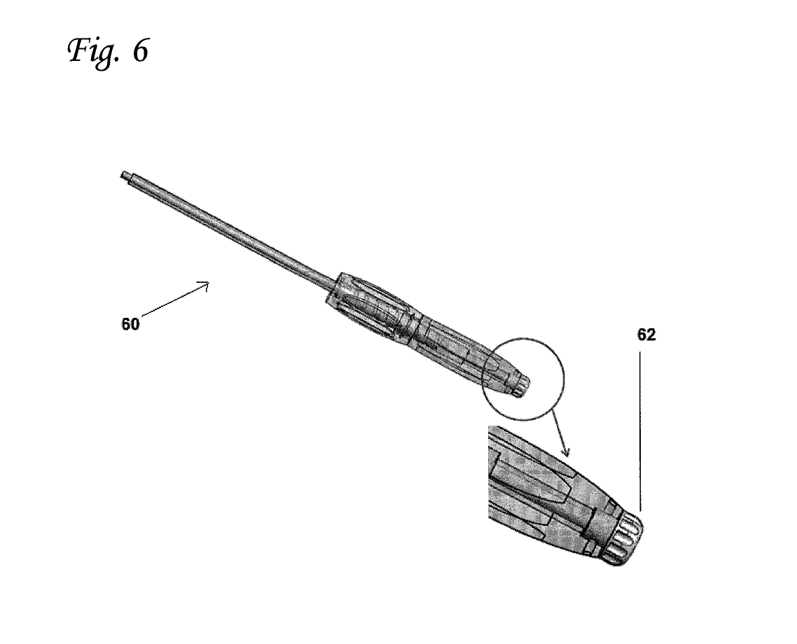

FIG. 6 illustrates a modification of the embodiment of the device shown in FIG. 5. In this case, the stabilizing element locking/release button 62 is located at the proximal extremity of the handle of delivery device 60.

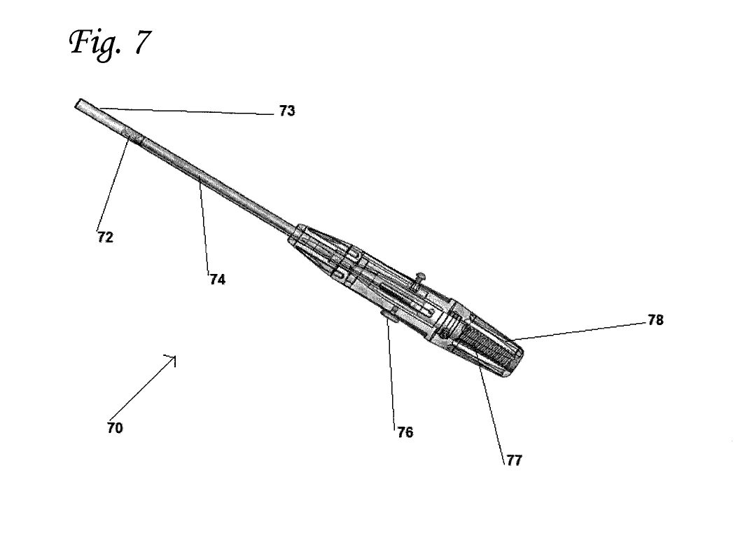

FIG. 7 illustrates another embodiment 70 of the delivery device of the present invention. It will be seen that a crimped valve support device 72 has been inserted into the lumen of the outer conduit 73 and is located along the longitudinal axis such that it is in contact with the inner tube 74. In this particular embodiment, the stabilizing element locking button 76 is located within the fixed central portion of the proximal handle, while the screw mechanism 77 for controlling the distal-proximal position of outer conduit 73 in relation to inner tube 74 is located within the rotatable control portion 78 at the proximal extremity of handle.

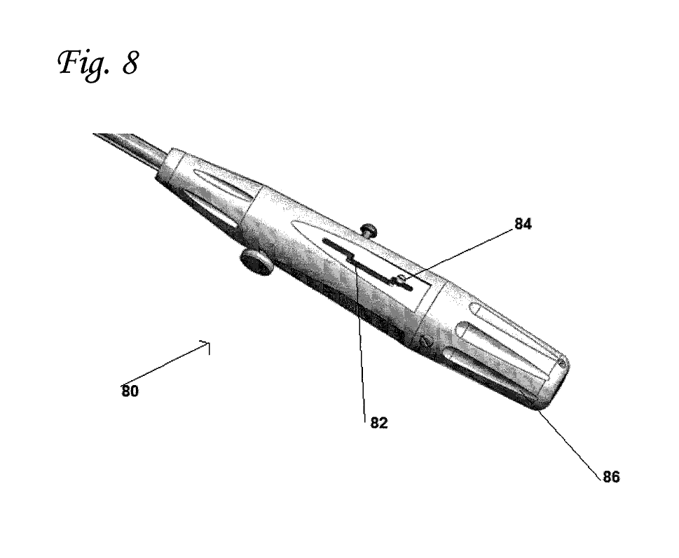

FIG. 8 illustrates a further embodiment of the delivery device of the present invention, wherein the proximal handle 80 incorporates (in its central, fixed portion) a slot 82 comprising a plurality of smaller slots that while continuous with each other, are not aligned along a single straight line. Rather, they are offset, thereby forming a series of ‘stops’ at the junction between each of said smaller slots and at the proximal and distal ends of the proximal and distal small slots, respectively. A marker pin 84 is provided within slot 82 said pin having a free lateral end which protrudes outside of said slot, and a medial end which engages with the internal screw mechanism that is used to control the distal-proximal position of the outer conduit. Thus, when the rotatable control portion 86 of handle 80 is rotated such that the outer conduit moves in relation to the inner tube, marker pin 84 is caused to move within slot 82. The operator is then able to determine at what stage the deployment procedure the valve support structure has reached (e.g. release of upper support element, release of bridging elements, release of lower support element) by reference to the position of marker pin 84 in relation to the aforementioned stop positions.

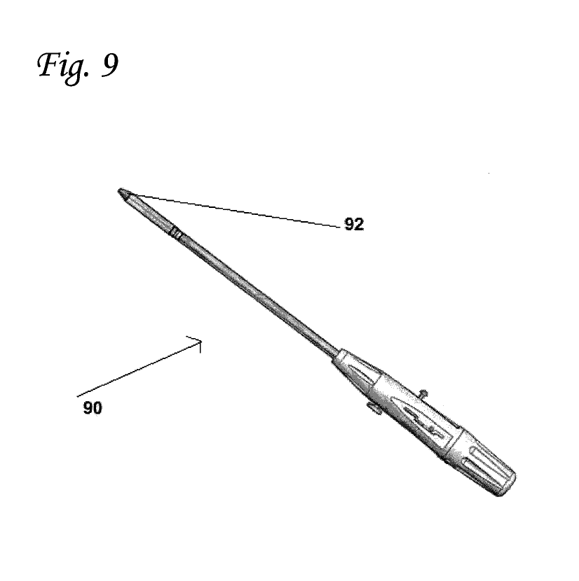

FIG. 9 depicts a delivery device 90 of the present invention that incorporates the position marker mechanism (i.e. slot and marker pin) described above and illustrated in FIG. 8. In addition, this embodiment also incorporates a distal tip 92 located at the distal tip of the device. Said tip assists the operator with the insertion and manipulation of the device through the various tissues encountered during delivery. The tip remains on the distal side of the valve support device, and following successful deployment thereof, is withdrawn proximally through the lumen of the said device (following its expansion) at the time that delivery device 90 is withdrawn from the body.

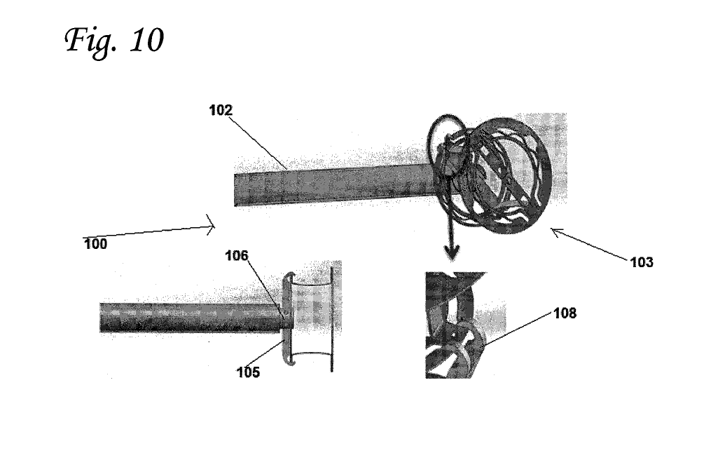

In all of the embodiments described hereinabove, the release of the valve support structure from the outer conduit is controlled by means of controlling the movement of said outer conduit in relation to the inner tube or rod. Thus, when the outer conduit is withdrawn proximally (or, alternatively, the inner tube is pushed distally), the crimped valve support device is released from the delivery device and then expands passively. FIG. 10, however, depicts a different embodiment of the invention, in which one of the support elements the valve support device is held by a jaw mechanism. Thus, in this figure, delivery device 100 comprises, in its distal part, an outer conduit 102, inside of which is inner tube 106. Pivotable jaws 105, which are attached to the distal end of said inner tube, grasp the lower ring of valve support device 103. As shown in the enlarged view in the bottom right corner of this figure, the jaws 105 have curved distal portions which are able to firmly hold the lower support ring of the valve support device.

In another embodiment of this device, not shown in the figures, said pivotable jaws are designed to grasp the upper (most distal) ring of the support device, and in case of a “single ring” support device, the pivotable jaws grasp the said single ring.

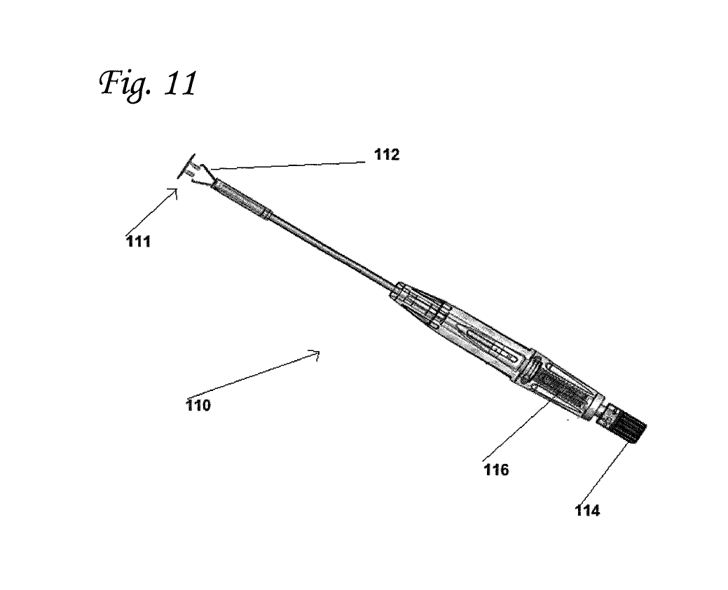

In the embodiment described immediately hereinabove (and illustrated in FIG. 10), the valve support device stabilizing elements are generally released when all, or most of, said support device has already been released from the delivery device. In certain circumstances, forces generated during release of the stabilizing elements, and their passive expansion may cause the support device to alter its position in relation to the valve annulus. In a further preferred embodiment, this potential drawback is solved by means of a dual mechanism, in which distally-located pivotable jaws (similar to those illustrated in FIG. 10) grasp the support device stabilizing elements, while at least one of the support elements are held by a wire to the control wire used to retain the stabilizing elements and control their release in the embodiments described hereinabove). A particularly advantageous feature of this embodiment is that it permits the pivotable jaws to be opened—thereby allowing the stabilizing elements to expand laterally—while the valve support device itself (i.e. the support ring thereof) is still being firmly held in place by means of the wire attached thereto. In this way, the stabilizing elements can be expanded without causing any displacement of the valve support device from its intended working position, thus allowing very accurate positioning and deployment of the support device. An example of this embodiment is presented in FIG. 11, which shows valve support device 111 being held at the distal end of delivery device 110. Although not shown in this drawing, the lateral stabilizing elements of valve support device 111 are maintained in their closed conformation by means of pivotable jaws 112, while the support ring in said support device is firmly held in place by means of a control wire. Independent control of the release of the stabilizing elements and of the support ring is achieved by use of the two rotatable controls situated on the proximal end of the handle, namely stabilizing element control 116 and valve support element control 114. With regard to the latter control, when the operator wishes to release the support device, control 114 is fully opened (and in some embodiments, completely removed) thereby allowing one end of the wire to be pulled out of the delivery device handle in a proximal direction. In this way the support device is released from the delivery device, and firmly anchored in position at the anatomical valve annulus by means of the stabilizing elements.

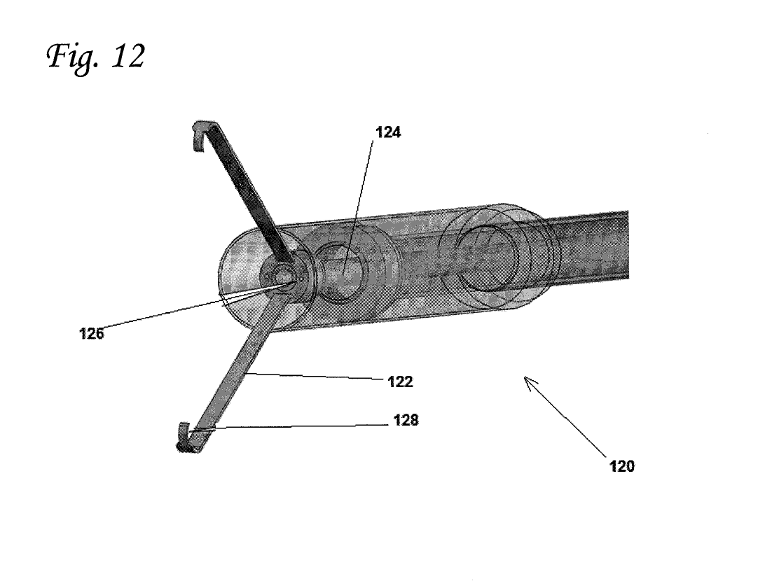

FIG. 12 provides an enlarged view of the distal end of delivery device 120, in which pivotable jaws 122 are mounted on a collar that is attached to the distal extremity of inner tube 124. This figure also shows two holes 126 formed in said collar, in order to allow the passage of the retaining/control wire. In addition, the curved tips 128 of the pivotable jaws (as described hereinabove) are also clearly seen in this view.

In one version of the embodiment described immediately hereinabove not shown in the figures), an additional conduit is used to maintain the pivotable jaws in a closed position, even after withdrawal of the outer conduit. In this version, the proximal handle further comprises a third rotatable (or other) control in order for the operator to be able to retract said additional conduit when he or she wishes to permit lateral expansion of the stabilizing elements.

As mentioned hereinabove, the outer conduit of the transapical embodiment of the present invention needs to be either rigid or semi-rigid, and may be manufactured from any suitable material including, but not limited to, biocompatible metals such as stainless steel or Nitinol, and biocompatible plastics or polymers such as Pebax, Nylon, Teflon or polyurethane. The outer conduit may be manufactured by any appropriate technique including extrusion, braiding and so on.

The proximal handle may be constructed from materials such as Delrin, Pebax, Nylon, Teflon, polyurethane and stainless steel or combinations thereof.

Generally, the outer conduit has a length in the range of 20-50 cm, preferably 30 cm, and an outer diameter in the range of 12-36 French, more preferably in the range of 18-24 French.

### Delivery Device for Transseptal Use:

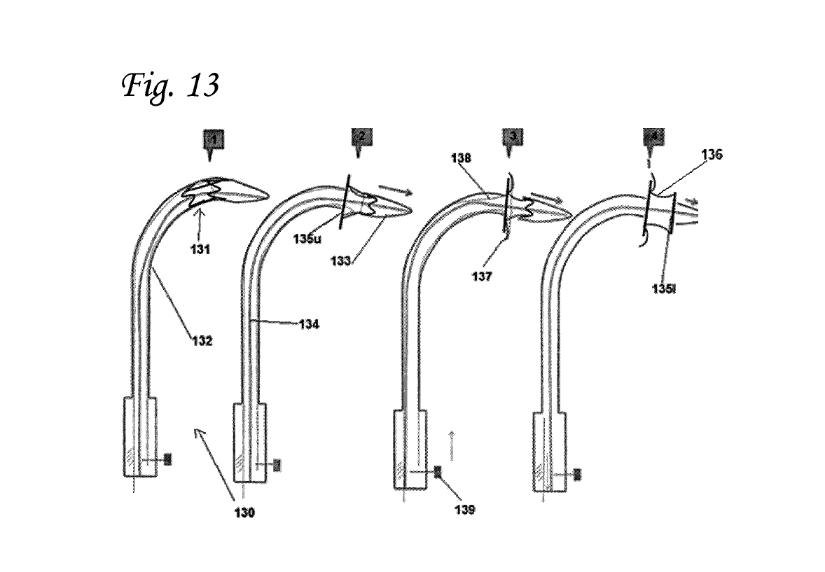

We now turn our attention to the embodiments of the present invention which are intended for use in the transseptal delivery of a cardiac valve support device. These embodiments are described in detail hereinbelow with reference to FIGS. 13 to 17.

In these embodiments, the delivery device comprises a proximal handle (similar to that described hereinabove in relation to the transapical embodiments) connected to and continuous with a distally-placed flexible catheter which is suitable in length and diameter for transfemoral vein entry over a guidewire.

An inner tube or guidewire is situated inside the catheter along its entire length and is connected at its distal end with a hollow distal tip. Said tip is fitted with a steering mechanism comprising two or more control wires attached thereto, as is commonly known to skilled artisans in this field.

As in the case of most of the transapical embodiments described above, a control/retention wire is used in order to retain the valve support device stabilizing elements in their closed, collapsed configuration until the operator decides to release them.

Generally, the proximal handle is fitted with a rotatable control, which when turned by the operator leads to lengthening or shortening of the inner tube or guidewire. In this way, the distance between the distal tip and the body of the delivery catheter can be controlled.

It should be noted that in the transseptal approach, the device is delivered “upside down”—i.e. the lower ring first, then the bridges and finally the upper ring.

FIG. 13 schematically depicts, in a generalized manner, the key features of the presently-disclosed transseptal delivery device 130, and its use in the delivery of a two-ring valve support device 131. Four separate representations of the device are shown, each of which depicts a different stage in the deployment of the valve support device. Thus, in stage 1 (at the extreme left of the figure), the valve support device 131 is entirely contained within the device—in the embodiment shown in the figure: partly within outer conduit 132, and partly within hollow distal tip 133. (In other embodiments, the crimped support device is contained entirely within the outer conduit.) In stage 2, the upper support element 135u has been released from the confines of the outer conduit 132 and has adopted its expanded configuration. Similarly, bridging elements 136 have also been partially released. At this stage, stabilizing elements 137 are still retained in their collapsed configuration by means of stabilizing element control wire 138. Next, in stage 3, the control wire release screw 139 is manipulated in order to release the end of the control wire that is attached thereto, thereby causing said wire to lose its tautness and permitting stabilizing elements 137 to expand laterally into their working configuration. Finally, in stage 4, stabilizing element control wire 138 is removed from the device, and the lower support element 135l and the remaining portion of the bridging elements 136 are released from the outer conduit and allowed to expand into their final working configuration.

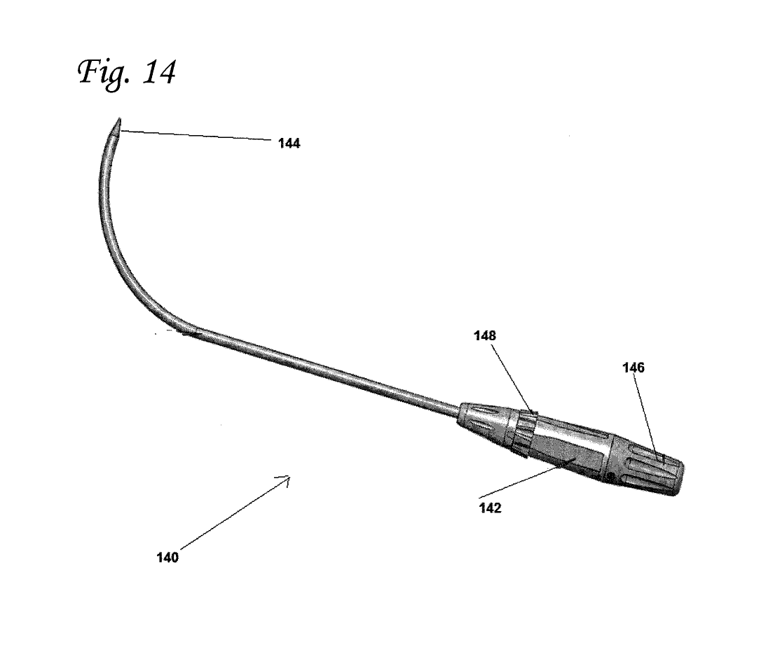

FIG. 14 presents an external view of an example of this embodiment of the device. It may be seen from this figure that delivery device 140 comprises a proximal handle 142 and an elongated catheter extending distally therefrom. A steerable hollow tip 144 is located at the distal end of the catheter. A rotatable deployment control 146—which is used to control the separation distance between the distal tip and the body of the catheter—is located proximally to the fixed portion 142 of the proximal handle, while a separate control ring 148, located on the distal side of said fixed handle portion is used to lock and/or release the retention wire used to maintain the valve support device stabilizing elements in their collapsed conformation.

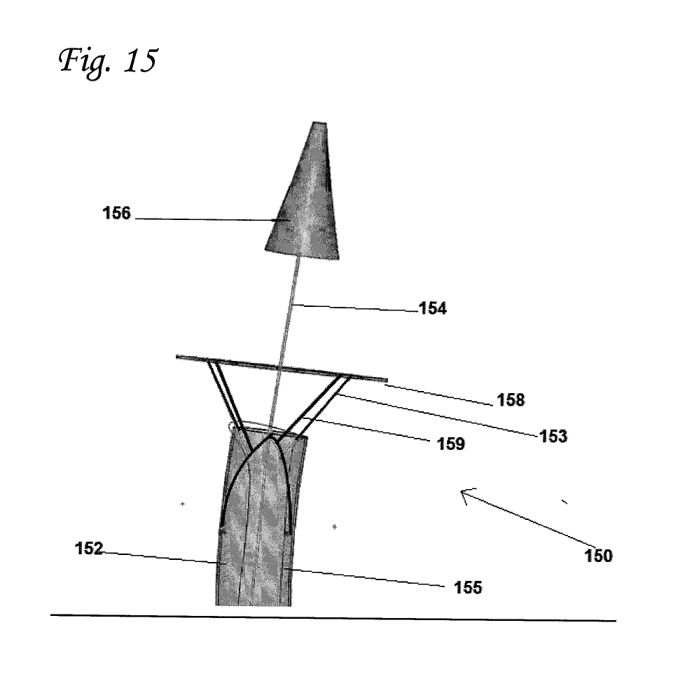

A more detailed view of the distal portion 150 of a transseptal delivery device after partial deployment of a cardiac valve support device according to the present invention is depicted in FIG. 15. It may be seen from this figure that distal tip 156 has been distanced from the catheter body 152, by means of moving said catheter body in a proximal direction, in relation to guidewire tube 154. As a result, the entire lower support ring 158 and most of the length of the bridging elements 159 have now been removed from the confines of said catheter body, leading to expansion of said lower support ring. The lateral valve support stabilizing elements 153 are maintained in their collapsed configuration by the presence of the taut retaining wire 155. It will be noted that in this embodiment of the invention, in which the valve support device is deployed transseptally (i.e. from above to below), the lower support ring is the first portion of the support device to emerge from the delivery device.

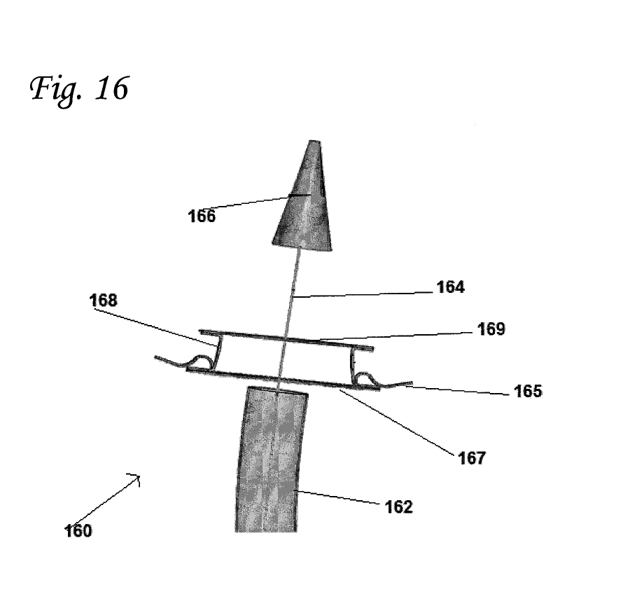

FIG. 16 shows the embodiment that was depicted in FIG. 15 at a later stage, after the separation distance between the distal tip 166 (mounted on the end of guidewire tube 164) and the catheter body 162 of the delivery device 160 has been further increased. As a result, the upper support ring 167 has now been released from said catheter body 162, and the entire support device—including lower support ring 169 and bridging elements 168—is now in its expanded, working conformation. In addition, the retaining wire has now been released and withdrawn proximally from the delivery device, thereby releasing stabilizing elements 165 and enabling them to expand laterally and contact the ventricular wall.

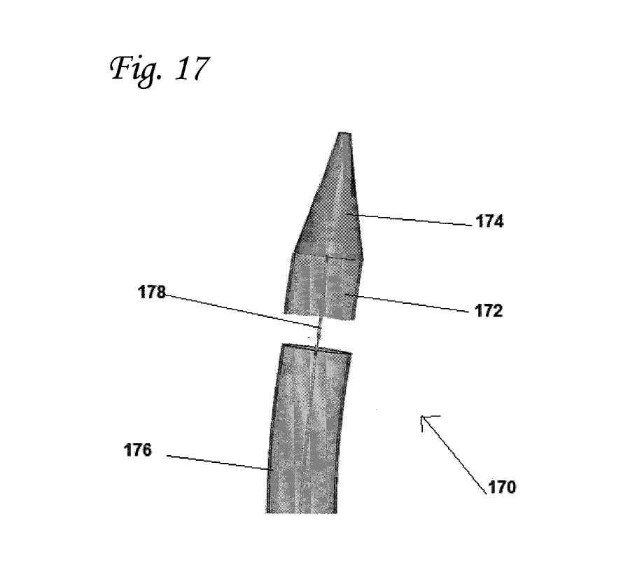

In some cases, it is desirable for the upper support ring to expand before the lower support ring. This may be achieved by means of the embodiment of the delivery device 170 shown in FIG. 17. This embodiment is characterized by the presence of a hollow cylindrical proximal extension 172 of the conical distal tip 174. In use, the cardiac valve support device is crimped and then inserted into the delivery device such that its distal portion (lower support ring) is contained within the aforementioned proximal extension of the distal tip, while its proximal portion (upper support ring) is contained within the catheter body 176. Thus, during deployment, the catheter body may be withdrawn proximally, in relation to the guidewire tube 178, thereby allowing the upper support ring to expand prior to the expansion of the lower support ring, which at that stage is still enclosed in its collapsed configuration within cylindrical tip proximal extension 172. Then, the stabilizing elements are allowed to expand laterally (by means of releasing the retaining wire). As a result, the upper support ring becomes anchored within the ventricular cavity, and further distal movement of the distal tip will cause the lower support ring to leave the confines of said tip and expand into its working configuration.

The transseptal delivery device of the present invention is generally passed over a guidewire through the femoral vein, in order to reach the right atrium. Then (in the case of mitral valve replacement), the delivery device passes across the atrial septum, thereby entering the left atrium, thus allowing the deployment of the valve support device in the region of the valve annulus. Other veins, such as the subclavian vein, may also be used as entry points for the delivery device into the circulatory system.

In order to be able to negotiate the circulatory system from the entry point puncture all the way to the target site within the heart, the catheter body needs to have an optimal degree of flexibility, such that, on the one hand, it may be steered around curved portions and junctions within the blood vessels (using the aforementioned steering wires), while on the other hand it may retain sufficient ‘pushability’ such that it does not buckle while being advanced towards the heart.

Preferably, the catheter body in the transseptal embodiments of the present invention is constructed from biocompatible polymers such as (but not limited to) Pebax, Nylon 12, Teflon and polyurethane. Standard techniques, well known to the skilled artisan in this field, such as extrusion may be used to manufacture the catheter body.

As in the case of the transapical embodiments, the proximal handle may be constructed from any suitable biocompatible plastic or polymer such as Delrin, Pebax, Nylon, Teflon, polyurethane and the like, or alternatively from a biocompatible metal such as stainless steel, or combinations of the aforesaid materials. Said handle may be manufactured using any suitable procedure including (but not limited to) injection molding, 3D printing, milling, CNC methods and so on.

Typically, the transseptal catheter body has a length in the range of 100 to 150 cm, preferably 115 cm. The outer diameter of said catheter body is generally in the range of 12-30 French preferably in the range of 18-24 French.

### Additional Embodiments Suitable for Either Transapical or Transseptal Use:

### Multiple Wire Embodiments:

In this embodiment of the delivery device of the present invention, the jaws that are used to control the deployment of the stabilizing elements which are present in some of the embodiments described hereinabove and described in the accompanying drawings (e.g. FIGS. 11 and 12) are replaced by control wires. Thus, in the presently-described implementation, both the stabilizing elements and the support elements are controlled by means of wires. This embodiment has been found, in some circumstances, to improve the reliability of the controlled release.

The wires may be constructed of any suitable material having the desired mechanical properties, including (but not limited to) Nitinol and stainless steel. In one preferred embodiment, the wires are constructed of Nitinol.

In one particularly preferred implementation of this embodiment of the delivery device of the present invention, the central rod (or inner tube) described hereinabove is replaced by a multi-lumen tube, wherein each of the control wires pass through a separate lumen, in order to prevent mutual entanglement. In addition, the use of separate channels for each wire improves the efficiency of their withdrawal at the end of the delivery procedure.

In one implementation of this embodiment, the delivery device comprises three separate control wires: one for each of the stabilizing elements and one for the cardiac valve support. Each wire passes from the proximal end of the device (i.e. the end that is held in the clinician's hand) through its own separate lumen, until lakes contact with either one of the stabilizing elements or the support ring. Each of said three wires then doubles back through additional lumens ending within or beyond the proximal end of the delivery device. Thus, in one preferred embodiment, the inner multi-lumen tube comprises six separate control wire lumens. Preferably, the multi-lumen tube further comprises a central lumen which is used for passage of the delivery device guidewire.

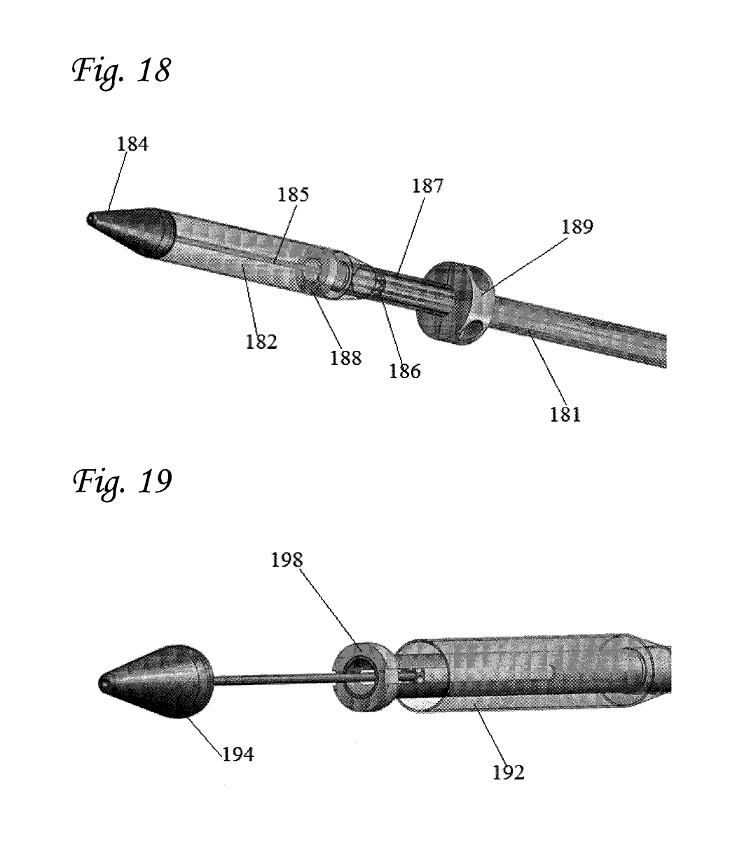

This implementation is illustrated in FIG. 18, which depicts a transapical delivery device in its pre-deployed state, and FIG. 19, which shows said device in its post-deployment configuration. Thus, it may be seen from FIG. 18 that prior to deployment of the valve support device at the cardiac valve annulus, the outer conduit 181 is in its distal-most position, such that the expanded distal capsular portion 182 of said conduit is in contact with distal tip 184, said tip being mounted on the distal end of guidewire tube 185. The inner multi-lumen tube 186, having a central guidewire lumen and six peripheral control wire lumens (of which four are visible) is contained within intermediate conduit 187. A collar 188 is fitted on the distal end of said intermediate conduit, said collar being fitted with grooves or recesses (not shown) into which the stabilizing elements are firmly held prior to deployment of the device. A movable marker ring and stopper 189 is also shown fitted around outer conduit 181, said ring being used to mark the depth of penetration of the transapical device within the heart.

Following deployment of the valve support device (i.e. following release of both the support ring and the stabilizing elements), said device appears as depicted in FIG. 19. It will be seen that the outer conduit has now been withdrawn proximally such that the distal end of the expanded capsular portion 192 of the outer conduit is now no longer in contact with the distal tip 194, thereby exposing collar 198, and thus enabling release of the stabilizing elements of the valve support device.

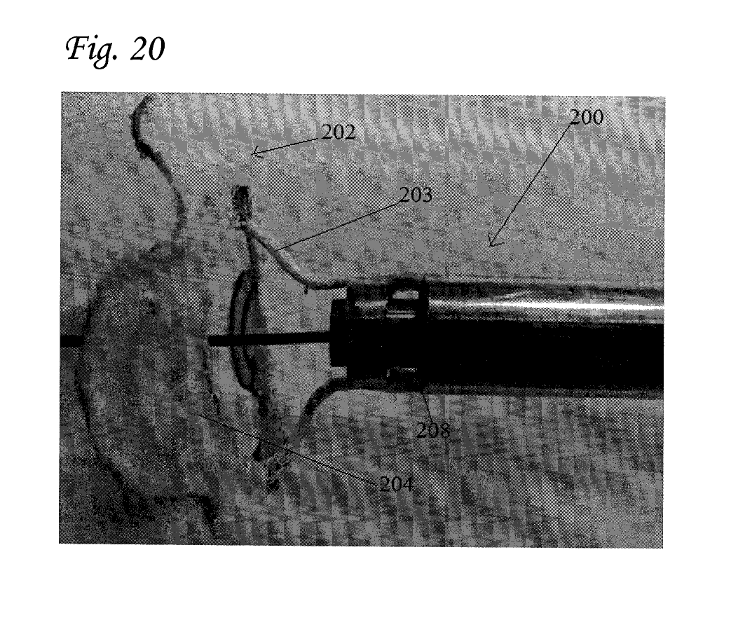

FIG. 20 is a photographic representation of the presently-described embodiment of the delivery device 200 with a single-ring valve support device 202 attached thereto, following release of the support ring 204. As shown, the free ends of each of the stabilizing elements 203 are held in place at the distal end of the device by collar 208.

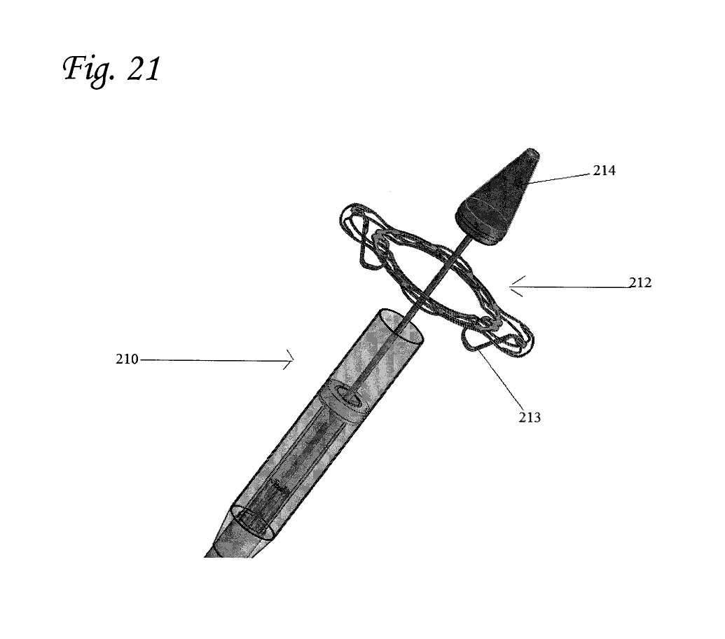

FIG. 21 illustrates single-ring valve support device 212 following its release from delivery device 210. It will be observed that the stabilizing elements 213 have been fully deployed such that they are able to exert upward and laterally-directed forces on the annular tissue. At this stage, all of the control wires will have been withdrawn proximally and removed from the delivery device. During the next (and final) stage, the entire delivery device will be withdrawn proximally from the body, the distal tip 214 of said device passing through the central cavity of the valve support device. In a variant of this procedure, the control wires are left in place after the delivery device has been removed.

In a particularly preferred embodiment, the multi-lumen tube may be manufactured from any suitable material, but is preferably selected from one or more of the following materials: Pebax, Nylon 12, PEEK, or any other biocompatible, medical-grade polymer. In a particularly preferred embodiment, the multi-lumen tube is constructed from Pebax. Typically, the multi-lumen tubing is manufactured using extrusion, but any other suitable method may also be employed.

In some preferred embodiments of this aspect of the present invention, the delivery system further comprises a mechanism for tensioning the central control wire (i.e. the wire that is used to stabilize the support ring during release of the anchoring elements). This mechanism is designed to maintain constant tension in the wire throughout all stages of the cardiac cycle, despite the movement of the heart muscle.

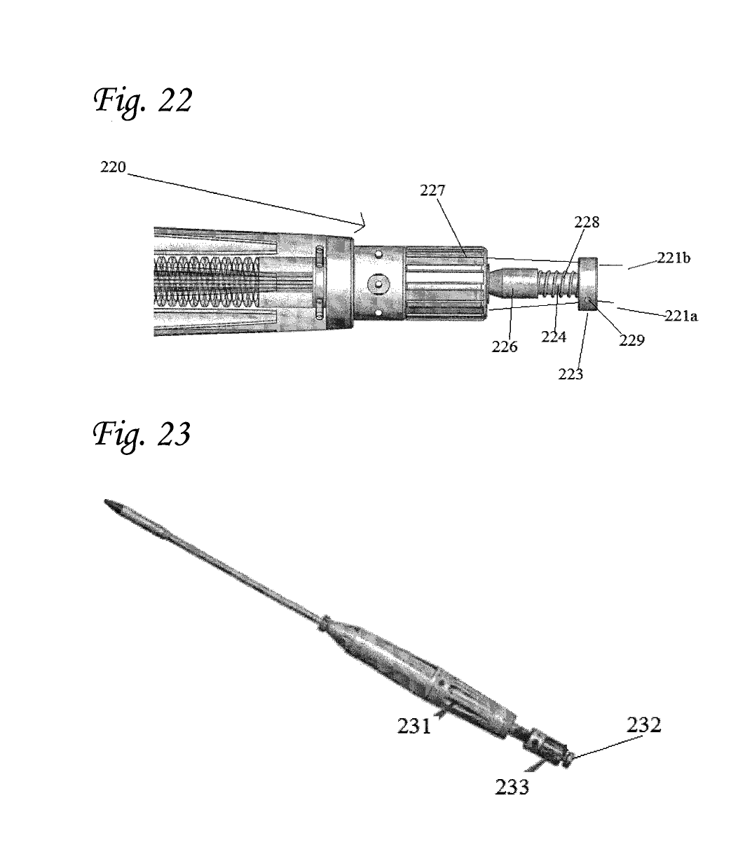

FIG. 22 illustrates the proximal end of one embodiment of the present invention that is fitted with an exemplary tensioning mechanism. It may be seen in this figure that both ends 221a and 221b of the support element control wire pass through separate apertures in the broadened head portion 223 of tensioning mechanism 220. Said head portion is continuous, distally, with a narrower plug 224 which is movable in a distal-proximal direction within an appropriately sized socket 226 that is fixed within an aperture formed in the proximal extremity of rotatable control sleeve 227. A helical spring 228 is fitted on the external surface of plug 224, such that proximal end of said spring is in contact with head portion 223, while the distal end thereof is enclosed within socket 226. Immediately prior to deployment of the stabilizing elements, the support element control wire is tensioned by means of securing its ends 221a and 221b Within the head portion of the tensioning mechanism by means of tightening small retaining screws (not shown) within radially-disposed threaded apertures 229 formed in the lateral surface of said head portion. Helical spring 228 then (as it lengthens) moves head portion 223 in a proximal direction, thereby tensioning the support element control wire.

The following section will describe the key stages of an exemplary method for implantation of a valve support device using the embodiment of the delivery system of the present invention. The various controlling elements operated by the clinician are indicated in FIG. 23:
1. Exposure of the support ring by means of withdrawing the outer tube in a proximal direction (i.e. in the direction of the operator), using distal rotatable control sleeve 231.
2. The support ring is then positioned within the patient's valve annulus.
3. The support ring control wire is locked in order to provide optimum support for the support ring. This is achieved by means of locking the ends of said wire in place by means of tightening the retaining screws in the distally-placed broad head 232 of the tensioning mechanism.
4. Distal control sleeve 231 is then further rotated in order to cause partial distal withdrawal of the anchoring wings from the confines of the delivery device.
5. The centrally-located multi-lumen tube is then advanced distally by means of proximal rotating control sleeve 233. In this manner, the anchoring wings are caused to become completely released from the delivery device, and passively expand into their fully open position.

It is to be emphasized that although this aspect of the invention has been described in detail (with the aid of FIGS. 18-23) with regard to a transapical delivery device, all of the various elements of the device and the procedure are readily adaptable for use in a transseptal procedure. The main adaption, in this regard, is the replacement of the rigid device body of the transapical device by a flexible catheter body in the case of the device intended for transseptal use.

### Mechanical Means for Controlling Deployment of Stabilizing Elements:

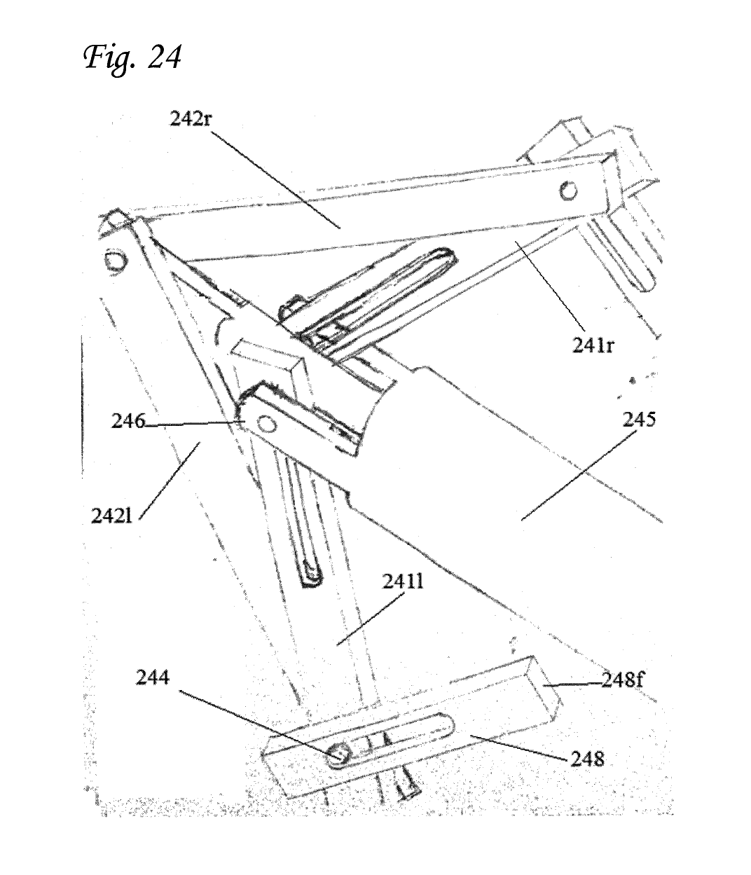

In another aspect, the present invention is also directed to a delivery system in which the wires that control the stabilizing elements (as described in the previous section) are replaced by a mechanical deployment mechanism. It is to be emphasized, however, that in this embodiment, the cardiac valve support ring is still controlled and supported by the central wire described hereinabove. This alternative mechanism represents an additional means for holding and supporting the support ring, while simultaneously allowing controlled unfolding of the anchoring wings. One particularly preferred implementation of this embodiment, in which the aforementioned deployment mechanism is provided in the form of a hinged four-sided assembly of interconnected short arms, comprising a pair of proximal arms and a pair of distal arms, the angles between two adjacent rods being alterable by means of operating a pusher tube that is attached to the pivotable junction between the two proximal arms. This preferred embodiment operates in a way that is similar to the familiar jack used to raise motor vehicles when changing tires at the roadside, with the exception that change in the angles between the arms in the present invention is caused by operating a pusher tube, rather than by operating a screw thread. This preferred embodiment will now be described with reference to FIGS. 24-28.

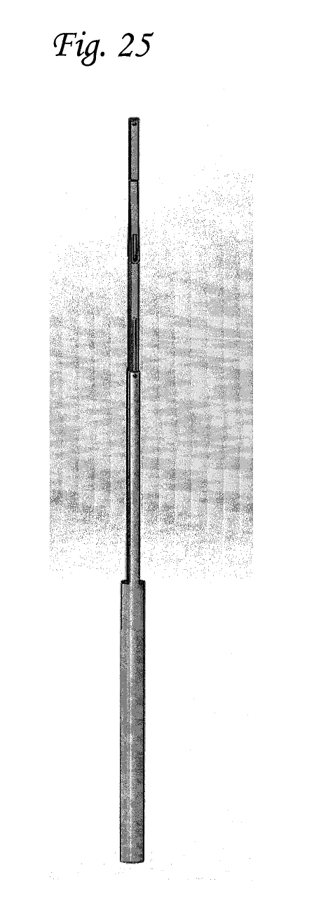

FIG. 24 provides an enlarged view of the aforementioned stabilizing element deployment mechanism, wherein said mechanism comprises a pair of proximal movable arms 241l and 241r, and a pair of distal movable arms 242l and 242r. Each arm is pivotably connected to the adjacent arm at a pivotable junction formed by a pin or rivet 244 passing through the ends of each adjacent pair of arms, such that the angles between the adjacent arms at each such pivotable junction may be altered. The means for altering these angles is provided by pusher tube 245 which surrounds the inner tube of the delivery device in a co-axial manner (and is similarly contained within the lumen of the outer conduit in a co-axial manner), and which ends in a strap-like bifurcation 246 The distal ends of each of said straps are connected by the aforementioned pins to the ends of proximal arms 241l and 241r. The lateral ends of the right and left proximal arms are connected to the corresponding lateral ends of the distal arms are similarly connected by pins or rivets at pivotable junctions. Two additional short arms 248 each having a first end and a second end are each attached at their first ends to one of these two junctions. Each of said short arms has a free end 248f to which the distal end of one of the stabilizing elements of the valve support device may be attached. Pusher tube 245 is connected at its proximal end to rotatable sleeve (233 in FIG. 23), such that upon rotation of said sleeve, said pusher tube is caused to move either proximally or distally (depending on the direction of the rotation). As shown in FIGS. 25-28, progressive distal movement of the pusher tube causes the angles at each of the pivotable junctions to change, thereby causing the position of the short arms (and hence of the stabilizing elements of the support device when attached thereto) to also change.

In the first stage (as shown in FIG. 25), the proximal movable arms and the distal movable arms are all co-aligned with the pusher tube along the long axis of the delivery device.

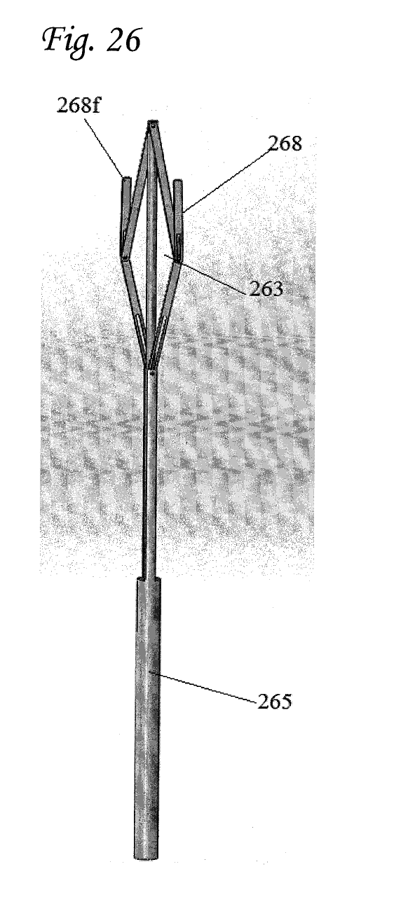

In the second stage (as shown in FIG. 26), pusher tube 265 has been advanced such that the angles between the adjacent pairs of distal and proximal arms have altered, such that they define an elongated rhombus 263. At this stage, the support ring (not shown) is released and, as a consequence of the increased separation distance between the free ends 268f of the two short arms 268 the stabilizing wings which are attached to said free ends are caused to partially open.

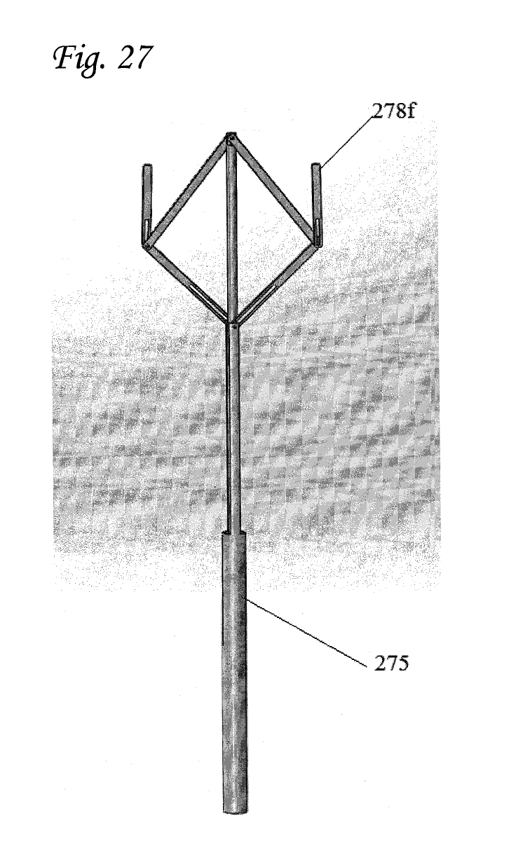

In the third stage (as shown in FIG. 27), there is further distal movement of the pusher tube 275, thereby causing the interconnected pairs of distal and proximal arms to adopt a broader, less elongated rhomboid shape, which in turn caused the free ends 278f of the short arms to further separate, thereby resulting in further opening of the anchoring wings.

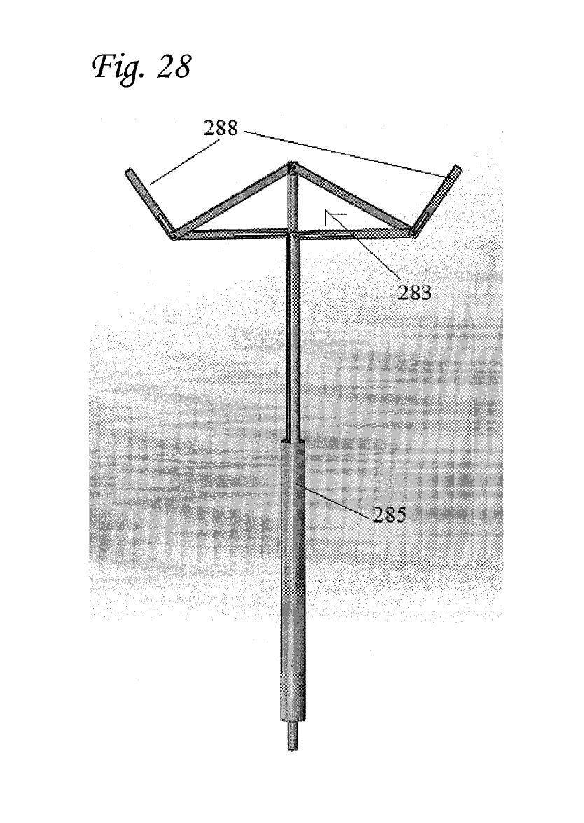

In the fourth stage (as shown in FIG. 28), there is further distal movement of the pusher tube 285, thereby causing the proximal and distal arms to define a triangular outline shape 283. The short arms 288 are now at their maximal mutual separation distance, and no longer apply any medially-directed forces on the stabilizing wings. Consequently, said wings may now be completely released from said arms.

Following the release of the stabilizing wings, the pusher tube may be advanced still further in a distal direction, thereby causing the distal arms and proximal arms to once again. adopt a parallel, in-line conformation, such that the delivery device may readily be withdrawn from the cardiac annulus, the perforation in the cardiac apex and the surgical entry wound.

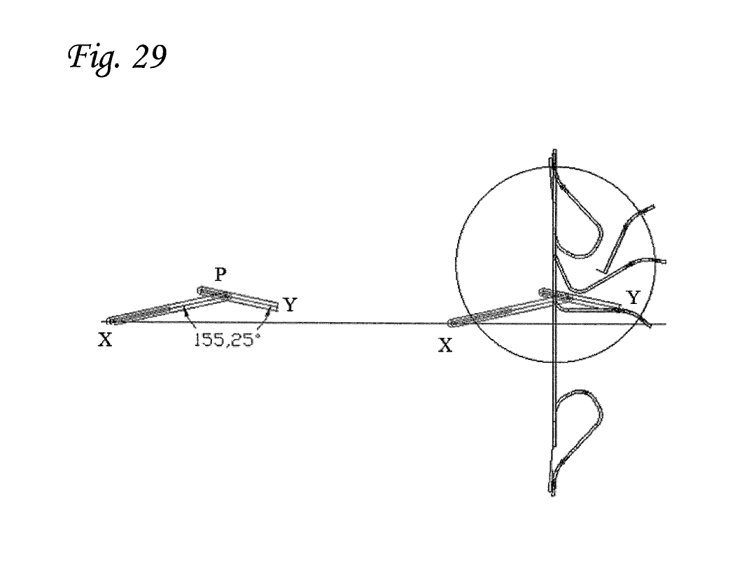

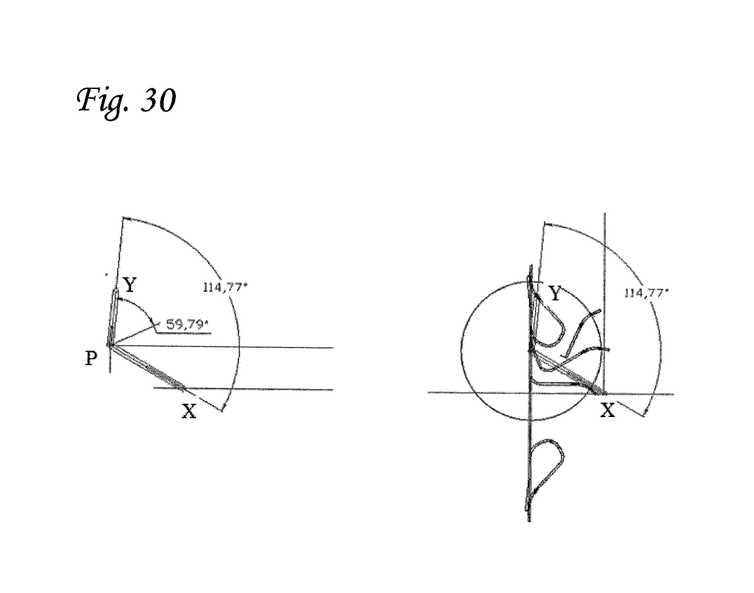

FIGS. 29 and 30 further illustrate the manner in which the free end of each short arm (to which the stabilizing wings are attached) is caused to change position, thereby resulting in the lateral rotation and “opening” of said wings into their working conformation. These figures were obtained following image analysis of photographic images obtained from inside the delivery device.

FIG. 29 illustrates the situation prior to activation of the “jack-like” mechanism described above, i.e. the mechanism when in its initial closed conformation (as shown in FIG. 25). Thus, the left-side of FIG. 29 depicts the angle between the short arm (represented by the length PY) and the proximal movable arm on the same side (represented by PX), wherein the point P represents the pivotable junction between the proximal arm, the distal arm (not and the short arm. The angle between the short arm and the proximal movable arm in this case is 155.25 degrees. The drawing on the right side of the figure shows the position of the stabilizing wing attached to the free end (Y) of the short arm when the device is in this initial conformation.

FIG. 30 illustrates the situation wherein the pusher tube has been moved distally such that the “jack-like” mechanism in its fully-open conformation (as depicted in FIG. 28). As shown in the left side of this figure, the short arm (PY) has now been rotated laterally, and the angle between it and the proximal movable arm (PX)—which has moved distally—is now 114.77 degrees. As shown in the right side of this figure, this altered geometry of the mechanism results in the lateral free end of the stabilizing wing being rotated laterally into its open, working position.

In addition to the aforementioned medially-directed forces exerted by the short arms on the stabilizing elements, this embodiment of the delivery device may further comprise additional mechanisms for retaining, and then releasing the stabilizing elements from the delivery system. Examples of such additional mechanisms include (but are not limited to):
1. Retaining pin that is released when the angle between the stabilizing wing and the free end of the short arm reaches a value, such that the geometries of said pin, said wing and said arm permits said pin to be easily removed from the device, and from the patient's body.
2. A locking mechanism comprising a releasable overtube that connects the stabilizing wing and the free end of the short arm.
3. A locking mechanism—for example a small ring or suture loop—that is capable of being released by means of being controllably broken.
4. A locking mechanism—such as a crescent shaped retaining clasp or partial ring that is capable of releasing a locking pin upon rotation.

The above-described jack-like mechanism may be constructed from one or more of the following materials: stainless steel, Nitinol, medical-grade polymers, and no on. In one preferred embodiment, the material used is Stainless steel 17.4 PH.

The above-described mechanism may be constructed using any of the techniques well known to the skilled artisan in the field, including (but not limited to) laser cutting, machining, 3D printing, erosion techniques, and so on.

Typically, the above-described mechanism, when in its closed conformation, has a diameter of 3.5 mm (i.e. the diameter of the pusher tube) and a length of 80 mm. In its open conformation, said mechanism has a diameter of 3.5 mm, a length of 90 mm and a width (i.e. between each of the short arms) of about 60 mm. These measurements are, of course, intended to be only examples of one preferred embodiment, and similar devices having different dimensions are also included within the scope of the invention.

The delivery device of the present invention may be constructed from any suitable biocompatible, medical-grade material including (but not limited to) stainless steel, Nitinol, Delrin, Pebax, Nylon 12, PEEK, and so on.

The device may be manufactured using any of the standard techniques well known to the skilled artisan in the field, including but not limited to: laser cutting, machining, 3D printing, erosion techniques and extrusion.

Generally, the device as an external diameter in the range of 8-50 mm, and a total end-to-end length in the range of 50-60 cm. These dimensions are given tor the sake of illustration only, and delivery devices having the essential features disclosed herein but with dimensions outside of these ranges, will, of course, be within the scope of the present invention.

1. A delivery device suitable for delivering a cardiac valve support, said valve support having at least one support element and a plurality of stabilizing elements,
wherein said delivery device comprises a proximal handle and an outer conduit that is continuous therewith; and
wherein said delivery device further comprises:
a) means for controlling the release of said support element from the open distal end of said outer conduit;
b) means for retaining said support device stabilizing elements in a closed conformation; and
c) means for independently controlling the release of said retaining means, thereby permitting the lateral expansion of said stabilizing elements.

2. The delivery device according to claim 1, wherein the means for controlling the release of the support element from the outer conduit comprise an inner tube or rod located within the lumen of the outer conduit, and a mechanism for moving the relative distal-proximal positions of said inner tube within the outer conduit.

3. The delivery device according to claim 2, wherein the mechanism causes the proximal movement of said outer conduit in relation to the inner tube or rod.

4. The delivery device according to claim 1, wherein the means for retaining the support device stabilizing elements in a closed conformation comprise a wire or thread that has a first end held within the proximal handle, wherein said wire or thread passes distally from said first end and through the support device and then passes proximally to a second end held within the proximal handle.

5. The delivery device according to claim 2, wherein the inner tube is a multi-lumen tube.

6. The delivery device according to claim 1, wherein the means for retaining the support device stabilizing elements in a closed conformation comprise two or more pivotable jaws attached to the distal end of the inner tube.

7. The delivery device according to claim 1, wherein the means for retaining the stabilizing elements in a closed conformation and the means for controlling the release of said stabilizing elements are provided by a laterally-expandable mechanism operated by a pusher tube, wherein said pusher tube is disposed co-axially with respect to the outer conduit.

8. The delivery device according to claim 7, wherein the laterally-expandable mechanism comprises:
a) two proximal movable arms and two distal movable arms joined together by means of pivotable junctions between two adjacent arms, such that said movable arms are capable of defining a quadrilateral outline shape;
b) two or more stabilizing element attachment arms pivotably attached at one of their ends to at least two of said pivotable junctions, wherein each of said short arms is adapted for attachment of a valve support device stabilizing element to its free end;
wherein the pusher tube is connected at its proximal end to a control mechanism within the proximal handle that may be used to move said tube proximally and distally;
and wherein said pusher tube is connected at its distal end to the pivotable junction between the two proximal movable arms.

9. The delivery device according to claim 1, wherein the outer conduit is rigid or semi-rigid, and wherein said device is suitable for use in a transapical procedure.

10. The delivery device according to claim 1, wherein the outer conduit is flexible, and wherein said device is suitable for use in a transseptal procedure.

11. The delivery de according to claim 1, wherein said device is suitable for delivering a mitral valve support device to the anatomical mitral valve annulus.
